(19) United States
(12) Patent Application Publication
(10) Pub. No.: US 2010/0136059 A1
(43) Pub. Date: Jun. 3, 2010

(54) HYBRID OPERON FOR EXPRESSION OF COLONIZATION FACTOR (CF) ANTIGENS OF ENTEROTOXIGENIC ESCHERICHIA COLI

(75) Inventors: Michael Lebens, Hokerum (SE); Ann-Mari Svennerholm, Vastra Frolunda (SE); Joshua Tobias, Goteborg (SE)

Correspondence Address: TRASKBRITT, P.C., P.O. BOX 2550, SALT LAKE CITY, UT, 84110, US

(73) Assignee: Crucell Sweden AB, Stockholm (SE)

(21) Appl. No.: 12/452,454

(22) PCT Filed: Jul. 1, 2008
(86) PCT No.: PCT/EP2008/058438

§ 371 Date: Dec. 29, 2009

(30) Foreign Application Priority Data
Jul. 2, 2007 (EP) 07111501.8

(51) Int. Cl. A61K 39/108; C07H 21/00; C12N 1/00; C12N 1/21; C12N 15/70; A61P 31/04

(52) U.S. Cl. 424/257.1; 536/23.7; 435/243; 435/252.3; 435/252.33; 435/476

(57) ABSTRACT

A recombinant operon comprising a gene assembly wherein there are at least two structural genes coding for at least two major subunits of colonization factor antigens (CFs) associated with enterotoxigenic Escherichia coli bacteria (ETEC), is disclosed. Further disclosed is a host cell, such as an Escherichia coli cell, genetically engineered to comprise such a recombinant operon, wherein said operon is located on an episomal element, such as a plasmid, or integrated in the chromosome of said host cell. Also disclosed is a method of producing a host cell capable of expressing from said operon at least two major subunits of colonization factor antigens (CFs) associated with enterotoxigenic Escherichia coli bacteria (ETEC). In addition, a vaccine composition against diarrhea comprising at least one such host cell together with pharmaceutically acceptable excipients, buffers, and/or diluents is disclosed. Finally is disclosed the use of said operon in the production of such a vaccine.

The present invention relates to hybrid operons for expression of colonization factor (CF) antigens of enterotoxigenic Escherichia coli, and in particular to a recombinant operon comprising genes including at least two structural genes for expression of two different CFs. The invention further relates to host cells comprising such an operon in the genome of the cell or in a plasmid inserted into the cell, such as an Escherichia coli cell.

### BACKGROUND OF THE INVENTION

Enterotoxigenic Escherichia coli (ETEC) is a major cause of travelers diarrhea and of diarrheal morbidity and mortality of children in endemic areas in many parts of the world. Virulence of the bacteria is associated with expression of fimbrial colonization factors (CFs) (Gaastra and Svennerholm, 1996) which mediate bacterial adhesion to the intestine and with secretion of heat-labile (LT) and/or heat-stable (ST) toxins which by affecting electrolyte and fluid transport processes in the gut are responsible for the diarrhea characteristic of the disease (Qadri et al, 2005a; Sanchez and Holmgren, 2005).

Protection against ETEC disease is associated with antibody-mediated neutralization of LT and immune responses against the CFs (Levine et al, 1994, Svennerholm and Holmgren, 1995; Svennerholm and Savarino, 2004). In general, the purpose of a vaccine is to induce an immune response in recipients that provides protection against subsequent challenge with the actual pathogen. This may be achieved by inoculation with a live attenuated strain of a pathogen, i.e. a strain having reduced virulence such that it does not cause the disease while still stimulating an effective immune response, or by administration of one or more killed strains of the pathogen that can elicit protective immune responses that are effective against infecting virulent strains. For immunization against enteric infections the vaccine should preferably be given by the oral route to efficiently stimulate an effective immune response locally in the intestinal mucosa, but also other mucosal routes or parenteral or even transcutanous routes may be used for inducing protective immunity.

Development of an effective vaccine that protects against disease caused by ETEC is difficult. More than 100 different serotypes have been associated with pathogenic strains. Furthermore, these strains can carry one or more of a large number of CFs (each of which is antigenically different) that facilitate the establishment of the infection in the intestine (Qadri et al, 2005a).

There is considerable evidence that immune responses directed against the CFs are protective, and that mucosal immune responses in the intestine are of particular importance for protection (Svennerholm et al, 1988, 1990; Levine et al, 1994; Svennerholm and Savarino, 2004). To induce such responses an ETEC vaccine should preferably be administered orally. We have previously developed an oral killed whole cell ETEC vaccine, containing five strains representing common ETEC serotypes and expressing several of the most commonly encountered CFs (in several cases usually referred to as coli surface [CS] proteins), i.e. CFA/I, CS1, CS2, CS3, CS4, and CS5 together with recombinant cholera toxin B subunit (CTB, which is highly homologous to the B subunit of ETEC LT) (Svennerholm and Holmgren, 1995; Svennerholm and Savarino, 2004). Initial clinical trials with this vaccine gave rise to significant immune responses against both CTB and the specific CFs present in the vaccine in Swedish volunteers and subsequently in adults and children in Egypt and Bangladesh (Jertborn et al, 1993; Ahren et al, 1998; Savarino et al, 1998, 1999, Qadri et al, 2005a, 2005b). The vaccine also provided significant protection against diarrhea sufficiently severe to interfere with the daily activity of American travelers going to Mexico and Guatemala (Sack et al, 2002; Svennerholm and Savarino 2004). However, the protection efficacy of the vaccine in Egyptian infants, 6-18 months of age was found to be low (Savarino et al, to be published). This suggested that whereas the vaccine was effective against more severe disease in travelers, it was not sufficiently potent to protect infants living in endemic areas (Svennerholm and Steele, 2004)

One of the reasons for the low efficacy of the described ETEC vaccine in infants is thought to be due to the comparatively low antibody responses found to the CF antigens in this age group (Savarino and Svennerholm, 2004). This poor response may be improved by giving higher dose of the different CFs, and hence increasing the amount of these antigens in a vaccine dose is a priority for continued development of a killed ETEC whole-cell vaccine. It is not feasible simply to increase the number of ETEC bacteria administered with each vaccine dose since it has been shown that giving high amounts of inactivated E. coli bacteria (even of an E. coli K12 placebo preparation) to young children 6-18 months of age can result in adverse reactions in the form of vomiting, probably due to the large amounts of endotoxin (LPS). These adverse effects were not observed if a lower (four-fold lower) dose of bacteria was given (Qadri et a/2005b, Savarino et al, to be published).

As is well known in the art, there are several types of CFs associated with human pathogenic strains of ETEC, but CFA/I, CFA/II and CFA/IV are the major types, currently associated with approximately 40-80% of clinical isolates. CFA/I is a single fimbrial antigen, whereas CFA/II and CFA/IV may be composed of more than one type of CF/CS proteins.

CF expression in wild-type ETEC appears to be restricted so that native strains only express a maximum of two or three types of CF antigens and then in certain combinations. Thus, native CFA/II ETEC strains generally express either CS1 together with CS3, CS2 with CS3 or CS3 alone. Similarly, native CFA/IV ETEC strains generally express CS4 with CS6, CS5 with CS6 or CS6 alone. However, e.g. CS1 and CS2 have not been found in the same wild type strain, and similarly CS4 and CS5 are not expressed together in naturally occurring strains. Furthermore, expression of CS4, CS5 or CS6 together with CS1 or CS2 or CS3 has not been described for wild type strains.

A minimum requirement that has been proposed for a vaccine against ETEC is that it should have the potential to induce protection against ETEC strains expressing CFA/I and the different subcomponents of CFA/II and CFA/IV. i.e. CS1-CS6. Thus, ETEC vaccines based on wild type strains may require a minimum of at least 5 bacterial strains, expressing CFA/I, CS1+CS3, CS2+CS3, CS4+CS6, and CS5+CS6.

One strategy to solve this problem was addressed in our pending International patent application PCT/SE2007/050051 where strains of E. coli which express elevated levels of ETEC CFs were used. The amount of these antigens can thus be increased in the vaccine without increasing the overall number of E. coli bacteria in the vaccine. This strategy was combined with insertion of at least one recombinant plasmid expressing an ETEC CF into a bacterial cell expressing another ETEC CF, thus providing an unnatural combination of expressed CFs from one bacterial strain.

The present application presents another solution to the problem of constructing an E. coli strain which provides expression of elevated numbers of ETEC fimbriae for use in vaccines against diarrhea.

### DESCRIPTION OF THE INVENTION

The present invention provides, in one aspect a recombinant operon comprising a gene assembly wherein there are at least two structural genes coding for at least two major subunits of colonization factor antigens (CFs) associated with enterotoxigenic Escherichia coli bacteria (ETEC). It is thus possible to express from this operon at least two structural genes coding for at least two major subunits of CFs, which enables reduction of the number of bacterial cells needed in a vaccine composition.

In another aspect of the invention a host cell is genetically engineered to comprise the above mentioned recombinant operon wherein said operon is located on an episomal element or integrated in the chromosome of the said host cell.

In a first embodiment of the invention the episomal element in the host cell is a plasmid.

In a second embodiment of the invention the at least two major subunits of colonization factor antigens (CFs) are the same or different. If for example operon 1 of a CF comprises A, B, C and D, where B is the major subunit and operon 2 of another CF comprises A′, B′, C′ and D′, where B′ is the major subunit, then operon 1 and 2 can be combined to A, B, B′, C and D or A′, B′, B, C′ and D′. In both cases two different major subunits will be expressed from the same operon. If the two major subunits of colonization factor are the same the operon would be e.g. A, B, B, C, D if B is the major subunit. This could induce a greater immune response against the major subunit B from a smaller amount of cells. An other example of the order of the gene fragments would be, B, A, C, D and the operon with two structural genes of major subunits would be B, B, A, C, D or B′, B A, C, D, respectively. In case several structural genes coding for major subunits of CFs are included in the same operon, these would be named B″, B′″ etc. An example of this strategy is given below, where the structural gene coding for the major subunit of CS2 is included in the CFA/I operon resulting in expression of the hybrid fimbriae CFA/I+CS2.

In a third embodiment of the invention the CFs are selected from the group consisting of CFA/I, CS1, CS2, CS3, CS4, CS5, CS6, CS14, CS17, CS19 and putative colonization factor O71 (PCFO71). Of these CFA/1, CS1, CS2, CS4, CS14, CS17, CS19 and putative colonization factor O71 (PCDO71) have a similarly constructed operon. These are thus preferably combined with each other.

In a fourth embodiment of the invention the host cell expresses the at least two major subunits of CFs in the operon.

In a fifth embodiment of the invention the host cell is a viable microorganism selected from the group consisting of bacteria and unicellular eukaryotes. The bacteria may be of such species as Vibrio cholerae and Escherichia coli and the unicellular eukaryotes may be of such species as yeasts and in particular yeast species such as Saccharomyces cerevisiae, Schizosaccharomyces pombe and Pichia pastoris.

In a sixth embodiment of the invention the host cell is an E. coli cell.

In a seventh embodiment of the invention the host cell is a non-toxigenic E. coil cell.

In an eight embodiment of the invention the host cell does not express an antibiotic resistance gene.

In a ninth embodiment of the invention the host cell carries one or more complementable chromosomal deletions or mutations that are complemented by one or more plasmids. These chromosomal deletions may for instance be located where genes necessary for production of essential amino acids are located.

Another aspect of the invention concerns a method of producing a host cell carrying at least one recombinant plasmid capable of expressing at least two major subunits of colonization factor antigens (CFs) associated with enterotoxigenic Escherichia coli bacteria (ETEC), comprising the steps of assembling in an operon genes or gene fragments required for expression of a hybrid ETEC CF; a promoter that controls the expression of the subunits; either integrating the operon into the genom of the host cell or transforming the host cell with a plasmid comprising the operon, a selection marker for plasmid maintenance and an origin of replication.

Host cells generated according to the invention can be used to manufacture a vaccine against ETEC diarrhea.

Thus, yet another aspect of the invention is directed to a vaccine against diarrhea comprising at least one host strain according to the invention together with pharmaceutically acceptable excipients, buffers and/or diluents, such as those selected for oral delivery of the vaccine. Suitable excipients, buffers and/or diluents for a vaccine can be found in the European or US pharmacopoeia.

In a final aspect of the invention there is provided the use of an operon according to the present invention in the production of a vaccine.

Since the described methods avoid the previous limitations of CF antigen expression in certain naturally occurring combinations, the invention may provide a vaccine against diarrhea comprising as few as 1-2 host strains which together express the major subunits of CFA/I, CS1, CS2, CS4, i.e. at least two major subunits of CFs by each strain. Thus, the vaccine will in total comprise of fewer strains, perhaps with the added advantage of being able to use lower doses of each strain than in earlier tested killed ETEC vaccines.

The expressed CFs contemplated for the purpose of vaccine production according to the invention are associated with ETEC causing intestinal infection and disease in mammals, especially humans.

Preferably the cells according to the invention express said CFs on the host cell surface.

The expression level obtained with the invention of CFs on the surface of host cells can be detected by an immunological method, e.g. by applying an inhibition ELISA assay.

In an embodiment of the invention, the major subunits of CFs that are expressed by a cell of the invention are expressed in a form that allow them to react with specific antibodies raised against corresponding major subunits of CFs from ETEC strains originally isolated from the stool of a mammal with intestinal ETEC infection.

In another embodiment of the invention, the CFs that are expressed by a cell of the invention are expressed in a form that when the cells are used in an effective amount for immunization of a mammal, leads to formation of antibodies against the expressed major subunits of CFs that can react with corresponding major subunits of CFs from ETEC strains originally isolated from the stool of a mammal with intestinal ETEC infection.

In yet another embodiment of the invention, the hybrid CFs that are expressed by a cell of the invention are expressed in a form that after inactivation of the cell by formalin treatment or other means, allows them to react with specific antibodies raised against corresponding subunits of CFs from ETEC strains originally isolated from the stool of a mammal with intestinal ETEC infection.

In still another embodiment of the invention, the CFs that are expressed by a cell of the invention are expressed in a form that after inactivation of the cell by formalin treatment or other means, when the cell is used in an effective amount for immunization of a mammal, leads to formation of antibodies against the expressed CFs that can react with corresponding CFs from ETEC strains originally isolated from the stool of a mammal with intestinal ETEC infection.

Host cells according to the invention are cultured by methods for in vitro culturing of the cells in liquid media providing expression of said hybrid CFs.

A cultured cell of the invention may be inactivated by using mild treatment with formalin or phenol or other means, thereby preventing the cell from replication, and resulting in a cell that retains the expressed hybrid CFs in essentially the same amounts (at least 50% of the original amount), and with essentially the same reactivity with antibodies and almost the same immunogenicity as for the cell before the inactivation.

One or several of the host cells of the invention is (are) especially suitable for use in a method of vaccinating a mammal against diarrhea, which comprises administering to the mammal a strain or combination of cells according to the invention.

In an embodiment of the invention, one or several of the host cells of the invention is (are) used alone or in combination as a vaccine, for vaccination of a mammal, such as a piglet, a calf, a lamb or a horse, or in particular a human being. Such a vaccine is preferably administered by the oral route.

The invention will now be illustrated by the following description of the drawing, the drawing, the sequence listing, the Materials and Methods and the Examples as well as the Table, but it should be understood that the invention is not limited to any disclosed details.

### DESCRIPTION OF THE DRAWING

FIG. 1 The FIGURE shows construction of the hybrid expression vector pJT-CFA/1-CS2(CotA). Using specific primers to amplify the CotA and the entire pJT-CFA/1-Amp, two fragments were amplified followed by ligation.

### MATERIALS AND METHODS

### Bacterial Strains and Culture Conditions

Strains described in this application are listed in Table 1. Construction of recombinant cells expressing a hybrid protein comprising two major subunits of members of a fimbriae family is exemplified by construction of a strain expressing the major subunits of both CFA/I and CS2.

Strains were kept frozen at −70° C. in a glycerol-containing freezing medium until used. After inoculation of an agar plate at 37° C. over night to ascertain growth and purity bacteria were grown in CFA broth (Casamino acids 10 g, Yeast extracts 1.5 g, MgSO₄7H₂O 102 mg, MnCl₂4H₂O 8 mg per liter), at 37° C. with shaking for 16-18 h. When necessary, the medium was supplemented with chloramphenicol (12.5 μg/ml) or ampicillin (100 μg/ml).

### Cloning of ETEC CFs Operon in Expression Vectors

### Production of CFA/I+CS2 as Hybrid Fimbriae

Production of a hybrid fimbriae that consists of the major subunit of CS2, and the usher, chaperon and the minor and major subunits of CFA/I, was done in several steps. A fragment containing the major subunit of CS2, CotA, was amplified by PCR using the Expand High Fidelity PCR System (Roche Diagnostics GmbH) the primers CS2-F-Hyb and CS2-R-Hyb (Table 1) and using the plasmid pJT-CS2-Cm (see pending patent application PCT/SE2007/050051) as template. Additionally, the plasmid pJT-CFA/1-Amp (See pending patent application PCT/SE2007/050051) was subjected to reverse PCR, using the primers CFA/1-F-Hyb and CFA/1-R-Hyb (Table 1), resulting in a fragment containing the original plasmid. Following restriction of both fragments with Eco31I, both fragments were ligated, resulting in a plasmid containing the entire operon of CFA/I and CotA downstream the CfaB.

### Expression of CFs

An over night culture of each recombinant TOP10 strain was diluted 1/100 in CFA broth, supplemented, with 100 or 12.5 μg/ml of ampicillin or chloramphenicol, respectively (Table 1), and incubated for 2 h at 37° C. and 150 rev/min, followed by addition of IPTG to the final concentration of 1 mM and incubation with the same conditions for additional 4 h. The bacteria were then harvested and re-suspended in PBS.

### Dot Blot Test

Specific monoclonal anti-CFAs antibodies were used to evaluate the expression of each CFAs on the cloned strains, as described previously (Binsztein et al 1991). Briefly, 2 μl of bacterial culture (10⁹ bacteria/ml in PBS) that have been washed with PBS, and induced with IPTG for expression CFA/I, were applied on the nitrocellulose filter papers and incubated with the MAbs followed by goat anti-mouse IgG, conjugated with HRP, for 1.5 h each. The final development was performed by 4-chloro-1-naphtol-H₂O₂ in TBS for up to 15 min.

### Hemagglutination

Fresh human or bovine erythrocytes were washed twice in 0.85% NaCl, suspended to 3% in saline with 1% D-mannose. Ten μl of this mixture, and the same volume of the tested bacterial suspension in PBS (10⁹ bacteria/ml) which were inducted for expression of the CFAs and washed with PBS, mixed and the hemagglutination was observed in 2 min at room temperature.

### Electron Microscopy

Ten μl of each bacterial suspension (10¹⁰ bacteria/ml in PBS), that had been washed once with PBS, were applied on parafilm. Formvar-coated grids were put on the suspension for 2 min. The grids were then washed twice, 10 sec each, by applying them on 25 μl of PBS-1% BSA on parafilm, followed by incubating the grids for 15 min with 25 μl specific monoclonal antibody diluted in PBS-Tween 0.05%-BSA 0.1%. The grids were washed 6 times with PBS-1% BSA, as above, and then incubated for 15 min with anti-mouse IgG-gold conjugate (Amersham International, Amersham, UK) in PBS-0.1% BSA-0.05% Tween. The grids were then washed three times with PBS-0.1% BSA, and three times with distilled water. Negative staining was performed by applying the grids on 25 μl of 1% ammonium molybdate (pH 7.0) for 50-60 sec, followed by air-drying the grids on a filter paper for 5 min. The grids were stored at 4° C. until examined by electron microscopy.

### ELISA

The amount of CFA/I or CS2 on the bacterial surface was quantified by an inhibition ELISA assay (Lopez-Vidal et al 1988), and the titers of IgA or IgG+M antibodies in serum determined by ELISA assay, as described previously (Rudin et al, 1994).

### Inactivation of Bacteria by Formalin

To kill the bacteria, the culture of each strain was washed and re-suspended with PBS to a density of 10¹⁰ bacteria/ml in PBS. Formalin was added to a final concentration of 0.1M, and the suspension incubated for 2 h at 37° C. and agitated with 60 rpm, followed by incubation of the suspensions at 4° C. for 3 days. The bacteria were then washed, re-suspended with the same volume of PBS, and 100 μl of the bacterial suspension was spread onto blood agar and incubated at 37° C. for up to a week to check for growth.

### Mice Immunization

Female Balb/c mice (6-8 weeks of age) were used for the immunizations in oral route. Cultures of induced and formalin killed reference strains 325542-3 and 58R957, and the recombinant strain TOP10-CFA/I-CS2, were washed and re-suspended in PBS to the desired bacterial density. 10⁹ bacteria together with 10 μg CT were used for immunization by the oral route, as previously described (Rhagavan et al, 200). All mice were given two identical immunizations two weeks apart, and bleedings were collected immediately before the first dose and two weeks after the second dose.

### Statistical Analysis

All ELISA and inhibition ELISA experiments were performed in duplicates and repeated at least three times on different days. Dot blot experiments for each particular test were repeated at least twice. Statistical analyses were conducted by the student's t-test and P<0.05 was regarded significant.

### EXAMPLES

### Example 1

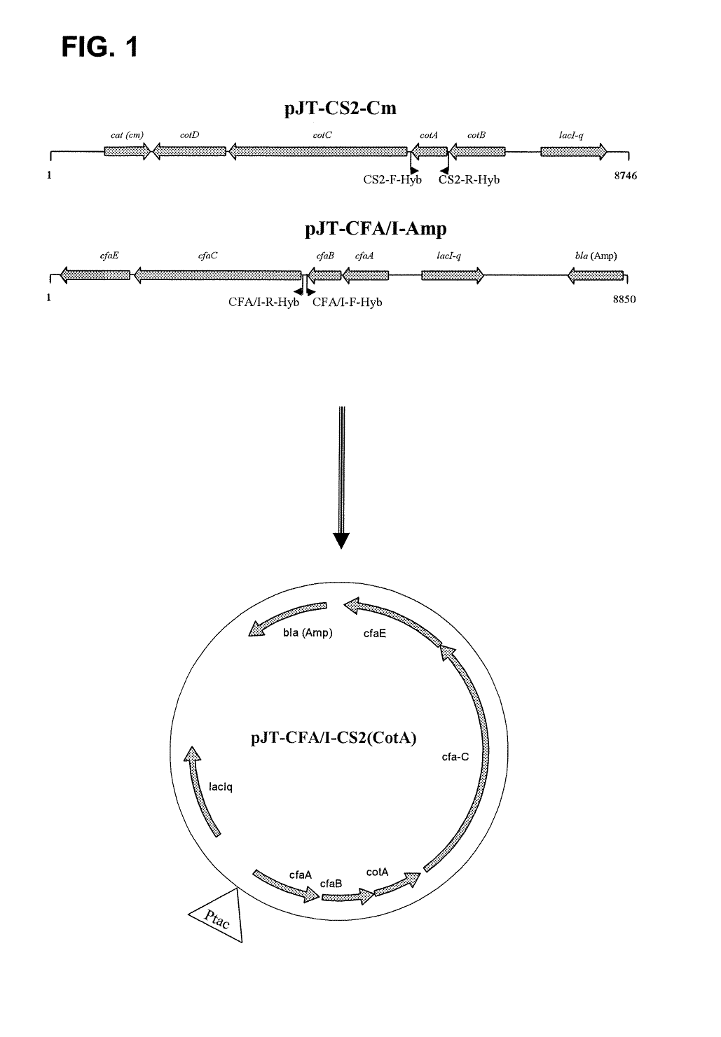

Production of hybrid fimbriae: We examined the possibility of expressing a hybrid fimbriae consisting of the major subunits of both CFA/I and CS2. Construction of an expression vector expressing such hybrid fimbriae is described in materials and methods, and depicted in FIG. 1. The vector was then propagated in Top10 strain, resulting in a recombinant strain expressing a fimbriae consisting of both major subunits of CFA/I and CS2. The expression was verified by using Transmission Electron Microscopy (TEM) and specific MAbs against each of major subunits, i.e. α-CFA/I MAb (1:6)˜goat α-mouse IgG 20 mn gold and Biotinylated α-CS2 MAb (10:3)˜Streptavidin 10 nm gold.

The teachings of references cited herein are hereby incorporated in this specification by reference.

TABLE 1
List of strains, plasmids, and primers used in this study.

| Strains, plasmid | | |
|---|---|---|
| and primers | Relevant characteristic | Source |
| Strains: | | |
| E. coli TOP10 | K12, F⁻ lambda⁻ | Invitrogen |
| TOP1O-CFA/I- | TOP10 expressing CFA/I and CS2(cotA) | This study |
| CS2(cotA)-Amp | | |
| ETEC 325542-3 | CFA/I ref. strain | |
| ETEC 278485-2 | CS2 ref. strain | |
| Plasmid: | | |
| pJT-CFAII-Amp | 8850 bp; amp^{r} | |
| pJT-CS2-Cm | 8472 bp; cm^{r} | |
| pJT-CFAII-CS2(cotA)- | | |
| Amp | | |
| Primers: | | |
| CFA/I-F | 5′-CGGTCTCGAATTCTGATGGAAGCTCAGGAGG | Acc. no. M55661 |
| SEQ ID NO: 1 | | |
| CFA/I-R | 5′-CGGTCTCAAGCTTTCTAGAGTGTTTGACTACTTGG | |
| SEQ ID NO: 2 | | |
| CS2-F | 5′-CGGTCTCGAATTCTTCTTGAAAGCCTCATGC | Acc. no. Z47800 |
| SEQ ID NO: 3 | | |
| CS2-R | 5′-CGGTCTCAAGCTTTTTACAGACTTGAACTACTAGG | |
| SEQ ID NO: 4 | | |
| CFA/I-F-Hyb | 5′-CGGTCTCTGCATTAAAGAATCAGGATCCCAAAGTC | |
| SEQ ID NO: 5 | | |
| CFA/I-R-Hyb | 5′-CGGTCTCTCATCTGGTATGTTTATACATCCCTC | |
| SEQ ID NO: 6 | | |
| CS2-F-Hyb | 5′-CGGTCTCTGATGTTTCTTTAATAACAGGGTGAC | |
| SEQ ID NO: 7 | | |
| CS2-R-Hyb | 5′-CGGTCTCTATGCTCAATAACCACTGTATAAGGG | |
| SEQ ID NO: 8 | | |

### REFERENCES

Ahren, C. M. Jertborn, and A. M. Svennerholm. 1998. Intestinal immune responses to an inactivated oral enterotoxigenic Escherichia coli vaccine and associated immunoglobulin A responses in blood. Infect. Immun. 66:3311-3316.
Binsztein, N., M. J. Jouve, G. I. Viboud, L. L. Moral, M. Rivas, I. Orskov, C. Ahrén, and A-M. Svennerholm. 1991. Colonization factors of enterotoxigenic Escherichia coli isolated from children with diarrhea in Argentina. J. Clin. Microbiol. 29:1893-1898.
Gaastra, W., and A. M. Svennerholm. 1996. Colonization factors of human enterotoxigenic Escherichia coli (ETEC). Trends Microbiol. 4:444-452.
Jertborn, M., C. Ahren, J. Holmgren, A. M. Svennerholm. 1993. Safety and immunogenicity of an oral inactivated enterotoxigenic Escherichia coli vaccine. Vaccine 16: 255-60.
Levine, M. M., J. A. Giron, and F. Noriega. 1994. Fimbrial vaccines. In Fimbriae: adhesion, biogenics, genetics and vaccines, P. Klemm (ed.), pp. 255-270. CRC Press, Boca Raton, Fla.
Qadri F., A. M. Svennerholm, A. S. G. Faruque, and Sack R. B. 2005a. Enterotoxigenic Escherichia coli in developing countries: epidemiology, microbiology, clinical features, treatment, and prevention. Clin. Microbial. Rev. 18:465-483.

Qadri, F., T. Ahmed, F. Ahmed, Y. A. Begum, D. A. Sack, A. M. Svennerholm and the PTE Study Group. 2005b. Reduced doses of oral killed enterotoxigenic Escherichia coli plus cholera toxin B subunit vaccine is safe and immunogenic in Bangladeshi infants 6-17 months of age: Dosing studies in different age groups. Vaccine 24:1726-1733.
Rhagavan S, M. Hjulström, J. Holmgren and A. M. Svennerholm. 2002. Protection against experimental Helicobacter pylori infection after immunization with inactivated H. pylori whole cell vaccines. Infect. Immun. 70:6383-6388.
Rudin, A., M. M. McConnell, A. M. Svennerholm. 1994. Monoclonal antibodies against enterotoxigenic Escherichia coli colonization factor antigen I (CFA/I) that cross-react immunologically with heterologous CFAs. Infect. Immun. 62:4339-4346.
Sack, D. A., J. Shimko, O. Torres et al. 2002. Safety and efficacy of a killed oral vaccine for enterotoxigenic E. coli diarrhea in adult travelers to Guatemala and Mexico. 42nd Interscience Conference on Antimicrobial Agents and Chemotherapy. San Diego, Calif.
Sánchez J., and J. Holmgren. 2005. Virulence factors, pathogenesis and vaccine protection in cholera and ETEC diarrhea. Curr. Opin. Immunol. 17:388-98.
Savarino, S. J., F. M. Brown, E. Hall, S. Bassily, F. Youssef, T. Wierzba, L. Peruski, N. A. El-Masry, M. Safwat, M. Rao, M. Jertborn, A. M. Svennerholm, Y. J. Lee, and J. D. Clemens. 1998. Safety and immunogenicity of an oral, killed enterotoxigenic Escherichia coli-cholera toxin B subunit vaccine in Egyptian adults. J. Infect. Dis. 177:796-799.
Savarino, S. J., E. Hall, S. Bassily, F. M. Brown, F. Youssef, T. F. Wierzba, L. Peruski, N. A. El-Masry, M. Safwat, M. Rao, H. El Mohamady, R. Abu-Elyazeed, A. Naficy, A. M. Svennerholm, M. Jertborn, Y. J. Lee, and J. D. Clemens. 1999. Oral, inactivated, whole cell enterotoxigenic Escherichia coli plus cholera toxin B subunit vaccine: results of the initial evaluation in children. J. Infect. Dis. 179:107-14.
Svennerholm A.-M., Y. L. Vidal, J. Holmgren, M. M. McConnell, and B. Rowe. 1988. Role of PCF8775 antigen and its coli surface subcomponents for colonization, disease, and protective immunogenicity of enterotoxigenic Escherichia coli in rabbits. Infect. Immun. 56:523-528.
Svennerholm, A.-M., C. Wenneras, J. Holmgren, M. M. McConnell, and B. Rowe. 1990. Roles of different coli surface antigens of colonization factor antigen II in colonization by and protective immunogenicity of enterotoxigenic Escherichia coli in rabbits. Infect. Immun. 58:341-346.
Svennerholm, A.-M., and J. Holmgren. 1995. Oral vaccines against cholera and enterotoxigenic Escherichia coli diarrhea. Adv. Exp. Med. Biol. 371 B:1623-1628.
Svennerholm, A.-M., and S. J. Savarino. 2004. Oral inactivated whole cell B subunit combination vaccine against enterotoxigenic Escherichia coli. In New Generation Vaccines: 3rd ed. Eds: Levine M. M. et al., pp. 737-750, Marcel Decker, New York, N.Y.
Svennerholm, A.-M., and D. Steele. 2004. Progress in enteric vaccine development. In: Microbial-gut interactions in health and disease, Ed. M. Farthing. Best Pract. Res. Clin. Gastroenterol. 18: 421-445.

1. A recombinant operon comprising a gene assembly wherein there are at least two structural genes coding for at least two major subunits of colonization factor antigens (CFs) associated with enterotoxigenic Escherichia coli bacteria (ETEC).

2. A host cell genetically engineered to comprise a recombinant operon according to claim 1, wherein said operon is located on an episomal element or integrated in the chromosome of said host cell.

3. The host cell according to claim 2, wherein the operon is located on an episomal element that is a plasmid.

4. The host cell according to claim 2, wherein the at least two major subunits of colonization factor antigens (CFs) are different.

5. The host cell according to claim 2, wherein the CFs are selected from the group consisting of CFA/I, CS1, CS2, CS4, CS14, CS17, CS19 and putative colonization factor O71 (PCFO71).

6. The host cell according to claim 2, wherein said host cell expresses the at least two major subunits of CFs.

7. The host cell according to claim 2, wherein the host cell is a viable microorganism selected from the group consisting of bacteria and unicellular eukaryotes.

8. The host cell according to claim 7, wherein the host cell is an Escherichia coli cell.

9. The host cell according to claim 8, wherein said E. coli cell is a non-toxigenic E. coli cell.

10. The host cell according to claim 2, wherein said host cell does not express an antibiotic resistance gene.

11. The host cell according to claim 2, wherein said host cell carries one or more complementable chromosomal deletion(s) or mutation(s) that are complemented by one or more plasmid(s).

12. A method of producing a host cell capable of expressing at least two major subunits of colonization factor antigens (CFs) associated with enterotoxigenic Escherichia coli bacteria (ETEC), the method comprising the steps of
assembling, in an operon, genes or gene fragments required for expression of a hybrid ETEC CF; a ETEC promoter that controls the expression of the subunits; either integrating the operon into the genome of the host cell or transforming the host cell with a plasmid comprising the operon, a selection marker for plasmid maintenance and an origin of replication.

13. A vaccine composition against diarrhea comprising at least one host cell according to claim 2, together with pharmaceutically acceptable excipients, buffers, and/or diluents.

14. The vaccine according to claim 13, wherein the pharmaceutically acceptable excipients, buffers, and/or diluents are selected for oral delivery of the vaccine.

15. A method of producing a vaccine, wherein the improvement comprises:
utilizing the operon according to claim 1 in the production of the vaccine.

16. The host cell of claim 2, wherein the at least two major subunits of colonization factor antigens (CFs) are the same.

17. The host cell of claim 3, wherein the at least two major subunits of colonization factor antigens (CFs) are different.

18. The host cell of claim 3, wherein the CFs are selected from the group consisting of CFA/I, CS1, CS2, CS4, CS14, CS17, CS19 and putative colonization factor O71.

19. The host cell of claim 3, wherein the host cell expresses the at least two major subunits of CFs.

20. An isolated Escherichia coli comprising:
a recombinant operon comprising nucleic acids encoding at least two major subunits of colonization factor antigens (CFs) associated with enterotoxigenic Escherichia coli bacteria (ETEC) wherein the CFs are selected from the group consisting of CFA/I, CS1, CS2, CS4, CS14, CS17, CS19, and putative colonization factor O71,
wherein the recombinant operon is located on an episomal element or integrated into the chromosome of the E. coli and wherein the E. coli expresses the at least two major subunits of CFs.


SEQUENCE LISTING (8 sequences)

SEQ ID NO: 1 - DNA, 31 bp, Artificial sequence
  primer named CFA/I-F
cggtctcgaattctgatggaagctcaggagg

SEQ ID NO: 2 - DNA, 35 bp, Artificial sequence
  primer named CFA/I-R
cggtctcaagctttctagagtgtttgactacttgg

SEQ ID NO: 3 - DNA, 31 bp, Artificial sequence
  primer named CS2-F
cggtctcgaattcttcttgaaagcctcatgc

SEQ ID NO: 4 - DNA, 35 bp, Artificial sequence
  primer named CS2-R
cggtctcaagctttttacagacttgaactactagg

SEQ ID NO: 5 - DNA, 35 bp, Artificial sequence
  primer named CFA/I-F-Hyb
cggtctctgcattaaagaatcaggatcccaaagtc

SEQ ID NO: 6 - DNA, 33 bp, Artificial sequence
  primer named CFA/I-R-Hyb
cggtctctcatctggtatgtttatacatccctc

SEQ ID NO: 7 - DNA, 33 bp, Artificial sequence
  primer named CS2-F-Hyb
cggtctctgatgtttctttaataacagggtgac

SEQ ID NO: 8 - DNA, 33 bp, Artificial sequence
  primer CS2-R-Hyb
cggtctctatgctcaataaccactgtataaggg